(19) United States
(12) Patent Application Publication
(10) Pub. No.: US 2025/0271432 A1
(43) Pub. Date: Aug. 28, 2025

(54) METHODS OF DETERMINING A HEALTH STATUS OF A CAT BASED ON ONE OR MORE BIOMARKERS, AND METHODS OF TREATING A MORTALITY RISK IDENTIFIED BY THE HEALTH STATUS

(71) Applicant: SOCIETE DES PRODUITS NESTLE S.A., Vevey (CH)

(72) Inventors: SEBASTIEN HERZIG, Morges (CH); PHILIPP GUT, Geneve (CH); LORANE TEXARI, Morges (CH); PASCAL STEINER, St. Louis, MO (US); YUANLONG PAN, Chesterfield, MO (US)

(21) Appl. No.: 18/859,269

(22) PCT Filed: Apr. 27, 2023
(86) PCT No.: PCT/EP2023/061059

§ 371 Date: Oct. 23, 2024

Related U.S. Application Data
(60) Provisional application No. 63/363,759, filed on Apr. 28, 2022.

(51) Int. Cl. G01N 33/573; G01N 33/569; G01N 33/68; G01N 33/72; G01N 33/80; G01N 33/92

(52) U.S. Cl. CPC G01N 33/573; G01N 33/56972; G01N 33/6842; G01N 33/72; G01N 33/80; G01N 33/92; G01N 2333/9108; G01N 2333/91188; G01N 2333/9123; G01N 2800/60

(57) ABSTRACT

The present invention provides a method for determining a mortality risk and/or probability of healthy lifespan of a cat; said method comprising determining the level of one or more biomarker(s) in one or more samples obtained from the cat, wherein the one or more biomarker(s) is selected from white blood cell count, haemoglobin, serum urea nitrogen, serum AST, serum chloride, serum total bilirubin, serum globulin, red blood cell count, serum sodium, serum cholesterol, serum potassium, serum alkaline phosphatase, and/or serum GGT.

### FIELD OF THE INVENTION

The present invention relates to a method for determining the health status, in particular the mortality risk and/or probability of a longer healthy lifespan, of a cat based on biomarkers.

### BACKGROUND TO THE INVENTION

The ability to determine information regarding the health of a cat, is desirable to inform about the cat's general health and well-being. For example, the ability to determine information about the general health of a cat may be helpful in assessing whether the cat would benefit from a dietary or supplement-based intervention.

Chronological age is known to be a major indicator of general health status, with increasing chronological age associated with reduced health. However, depending on genetics, nutrition, and lifestyles, individuals may age slower or faster than their chronological age. Chronological age may therefore not always reflect an individual's rate of aging or risk of reduced health. On the other hand, the biological age of an individual (based on e.g. clinical biochemistry and cell biology measures) can vary compared to others of the same chronological age. Methods for determining biological age may be helpful for identifying individuals at risk of age-related disorders earlier than would be expected based on their chronological age (see e.g. WO2019/165064).

However, there is a need for further methods of determining information regarding the health of cats.

### SUMMARY OF THE INVENTION

The present invention relates to a method for quantifying the health status of a cat based on biomarkers, in particular blood biomarkers. The method enables a determination of mortality risk and/or probability of a healthy lifespan for a cat through assessment of the cat's phenotypic age.

Existing methods that use blood biomarkers to assess the health status of cats determine biological age based on correlations between blood biomarkers and chronological age. Calculating the biological age of an animal may comprise determining differences in the level of one or more biomarker(s) compared to the expected level of the biomarker(s) at a given chronological age. Such methods are therefore based on the use of chronological age as the primary indicator of overall health.

In contrast, the present invention takes into account the direct predictive value of the described biomarkers on mortality risk and/or probability of a healthy lifespan. By way of example, a given biomarker may not directly correlate with chronological age, but may be indicative of a particular pathological condition and thus an increased mortality risk and/or a probability of a reduced healthy lifespan. The present methods may thus be described as identifying the phenotypic age (PhenoAge) of a cat. As such, the biomarkers of the present invention do not necessarily correlate with chronological age, but are related to the difference between phenotypic and chronological age of the cat.

The present invention provides a method for determining a mortality risk and/or probability of a healthy lifespan of a cat; said method comprising determining the level of one or more biomarker(s) in one or more samples obtained from the cat.

In one aspect the present invention provides a method for determining a mortality risk and/or probability of a healthy lifespan of a cat; said method comprising determining the level of one or more biomarker(s) in one or more samples obtained from the cat, wherein the one or more biomarker(s) is selected from white blood cell count, haemoglobin, serum urea nitrogen, serum AST, serum chloride, serum total bilirubin, serum globulin, haematocrit, red blood cell count, serum albumin, and/or mean corpuscular hemoglobin concentration.

The one or more biomarker(s) may be selected from white blood cell count, haemoglobin, serum urea nitrogen, serum AST, serum chloride, serum total bilirubin, serum globulin, and/or red blood cell count.

The present inventors have shown that each of these biomarkers is individually correlated with phenotypic age and is thus predictive of mortality risk and/or probability of a healthy lifespan.

Determining a mortality risk may refer to determining a likelihood that a cat will live for a longer or shorter period of time compared to an equivalent cat of—for example—the same chronological age. Accordingly, the present methods may determine the probability of a health span or a healthy lifespan for a cat compared to an equivalent cat of—for example—the same chronological age. Suitably, mortality risk may be equated to the probability of a healthy lifespan for the cat; wherein a decreased mortality risk is equated to an increased probably of longer healthy lifespan for the cat or an increased mortality risk is equated to a decreased probability of longer healthy lifespan for the dog. The mortality risk may be represented as the difference between phenoage and chronological age (phenoage advance) of the cat. For example, an increase in phenoage compared to chronological age may be indicative of an increased mortality risk for the cat; whilst a decrease in phenoage compared to chronological age may be indicative of a decreased mortality risk for the cat.

In one embodiment, the method may comprise determining the white blood cell count in one or more samples obtained from the cat. The method may further comprise determining the level of one or more biomarkers selected from hemoglobin, serum urea nitrogen, serum AST, serum chloride, serum total bilirubin, serum globulin, serum sodium, serum cholesterol, serum potassium, serum alkaline phosphatase, serum GGT, and/or red blood cell count in one or more samples.

In one embodiment, the method may comprise determining the level of hemoglobin in one or more samples obtained from the cat. The method may further comprise determining the level of one or more biomarkers selected from white blood cell count, serum urea nitrogen, serum AST, serum chloride, serum total bilirubin, serum globulin, serum sodium, serum cholesterol, serum potassium, serum alkaline phosphatase, serum GGT, and/or red blood cell count in one or more samples.

In one embodiment, the method may comprise determining the level of serum urea nitrogen in one or more samples obtained from the cat. The method may further comprise determining the level of one or more biomarkers selected from white blood cell count, hemoglobin, serum AST, serum chloride, serum total bilirubin, serum globulin, serum sodium, serum cholesterol, serum potassium, serum alkaline phosphatase, serum GGT, and/or red blood cell count in one or more samples.

In one embodiment, the method may comprise determining the level of serum AST in one or more samples obtained from the cat. The method may further comprise determining the level of one or more biomarkers selected from white blood cell count, hemoglobin, serum urea nitrogen, serum chloride, serum total bilirubin, serum globulin, serum sodium, serum cholesterol, serum potassium, serum alkaline phosphatase, serum GGT, and/or red blood cell count in one or more samples.

In one embodiment, the method may comprise determining the level of serum chloride in one or more samples obtained from the cat. The method may further comprise determining the level of one or more biomarkers selected from white blood cell count, hemoglobin, serum urea nitrogen, serum AST, serum total bilirubin, serum globulin, serum sodium, serum cholesterol, serum potassium, serum alkaline phosphatase, serum GGT, and/or red blood cell count in one or more samples.

In one embodiment, the method may comprise determining the level of serum total bilirubin in one or more samples obtained from the cat. The method may further comprise determining the level of one or more biomarkers selected from white blood cell count, hemoglobin, serum urea nitrogen, serum AST, serum chloride, serum globulin, serum sodium, serum cholesterol, serum potassium, serum alkaline phosphatase, serum GGT, and/or red blood cell count in one or more samples.

In one embodiment, the method may comprise determining the level of serum globulin in one or more samples obtained from the cat. The method may further comprise determining the level of one or more biomarkers selected from white blood cell count, hemoglobin, serum urea nitrogen, serum AST, serum chloride, serum total bilirubin, serum sodium, serum cholesterol, serum potassium, serum alkaline phosphatase, serum GGT, and/or red blood cell count in one or more samples.

In one embodiment, the method may comprise determining the serum sodium level in one or more samples obtained from the cat. The method may further comprise determining the level of one or more biomarkers selected from white blood cell count, hemoglobin, serum urea nitrogen, serum AST, serum chloride, serum total bilirubin, serum globulin, serum cholesterol, serum potassium, serum alkaline phosphatase, serum GGT, and/or red blood cell count in one or more samples.

In one embodiment, the method may comprise determining the serum cholesterol in one or more samples obtained from the cat. The method may further comprise determining the level of one or more biomarkers selected from white blood cell count, hemoglobin, serum urea nitrogen, serum AST, serum chloride, serum total bilirubin, serum globulin, serum cholesterol, serum potassium, serum alkaline phosphatase, serum GGT, and/or red blood cell count in one or more samples.

In one embodiment, the method may comprise determining the level of serum potassium in one or more samples obtained from the cat. The method may further comprise determining the level of one or more biomarkers selected from white blood cell count, hemoglobin, serum urea nitrogen, serum AST, serum chloride, serum total bilirubin, serum globulin, serum cholesterol, serum alkaline phosphatase, serum GGT, and/or red blood cell count in one or more samples.

In one embodiment, the method may comprise determining the level of serum alkaline phosphatase in one or more samples obtained from the cat. The method may further comprise determining the level of one or more biomarkers selected from white blood cell count, hemoglobin, serum urea nitrogen, serum AST, serum chloride, serum total bilirubin, serum globulin, serum cholesterol, serum potassium, serum GGT, and/or red blood cell count in one or more samples.

In one embodiment, the method may comprise determining the level of serum GGT in one or more samples obtained from the cat. The method may further comprise determining the level of one or more biomarkers selected from white blood cell count, hemoglobin, serum urea nitrogen, serum AST, serum chloride, serum total bilirubin, serum globulin, serum cholesterol, serum potassium, serum alkaline phosphatase, and/or red blood cell count in one or more samples.

In one embodiment, the method may comprise determining the red blood cell count in one or more samples obtained from the cat. The method may further comprise determining the level of one or more biomarkers selected from white blood cell count, hemoglobin, serum urea nitrogen, serum AST, serum chloride, serum total bilirubin, serum globulin, serum cholesterol, serum potassium, serum alkaline phosphatase, and/or serum GGT in one or more samples.

The method may comprise determining the level of each of white blood cell count, serum haemoglobin, serum urea nitrogen and serum AST.

The method may comprise determining the level of one or more further biomarkers selected from haematocrit, red blood cells count, serum albumin and/or mean corpuscular hemoglobin concentration in the one or more samples obtained from the cat.

In one embodiment, the method comprises determining the level of each of white blood cell count, haemoglobin, serum urea nitrogen, serum AST, serum chloride, serum total bilirubin, serum globulin, haematocrit, red blood cells count, serum albumin and mean corpuscular hemoglobin concentration.

In one embodiment, the present invention provides a method for determining a mortality risk of and/or probability of a healthy lifespan a cat; said method comprising:
a. determining the level of the following biomarkers; white blood cell count, haemoglobin, serum urea nitrogen, serum AST, serum chloride, serum total bilirubin, serum globulin, red blood cell count, serum sodium, serum cholesterol, serum potassium, serum alkaline phosphatase, and serum GGT in one or more samples obtained from the cat; and
b. determining a phenotypic age (Phenoage) of the cat using formula (1):

$$Phenoage = ln ( \frac{{γ}_{2} * {e}^{xb} * \{ {e}^{γ * age} - 1 \}}{{e}^{{β}_{02}} * γ} + 1 ) * \frac{1}{{γ}_{2}}$$

where xb is the sum of the value of each biomarker(s), multiplied by their respective coefficients according to formula (2):

$$xb = {\sum }_{u = 1}^{p} {x}_{u} {β}_{u} + {β}_{0}$$

and wherein the phenotypic age is used to determine a mortality risk and/or probability of a healthy lifespan for the cat.

Suitably, determining that the phenoage of the cat is greater than its chronological age is indicative of a higher mortality risk. Suitably, determining that the phenoage of the cat is less than its chronological age is indicative of a reduced mortality risk. Suitably, determining that the phenoage of the dog is greater than its chronological age is indicative of a reduced probability of a longer healthy lifespan. Suitably, determining that the phenoage of the dog is less than its chronological age is indicative of an increased probability of a longer healthy lifespan.

In one embodiment, the one or more samples is a blood sample, e.g. a blood plasma sample.

The invention further relates to a method for selecting a lifestyle or dietary regime for a cat, the method comprising:
a. performing the method as described in the first aspect of the invention; and
b. selecting a suitable lifestyle or dietary regime based on the mortality risk and/or probability of a healthy lifespan determined in step a.

The invention further provides a method for determining the efficacy of a lifestyle or dietary regime for reducing the mortality risk of a cat, said method comprising: a. performing the method as described in the first aspect of the invention; b. applying a change in lifestyle or dietary regime to the cat; c. performing the method as described in the first aspect of the invention after a time period of applying the change in lifestyle or dietary regime; and d. determining if there has been a change in mortality risk for the cat between step a. and step c.

The invention further provides a dietary intervention for use in reducing the mortality risk and/or probability of a healthy lifespan of a cat, wherein the dietary intervention is administered to a cat with a mortality risk determined by the method of the present invention.

The invention also provides the use of a dietary intervention to reduce the determined mortality risk and/or probability of a healthy lifespan of a cat, wherein the dietary intervention is administered to a cat with a mortality risk and/or probability of a healthy lifespan determined by the method of the present invention.

The invention further provides a computer-readable medium comprising instructions that when executed cause one or more processors to perform the method of the present invention.

The invention also provides a computer system for determining a mortality risk and/or probability of a healthy lifespan of a cat, the computer system programmed to perform one or more of the steps of: determine the mortality risk and/or probability of a healthy lifespan of a cat given the level of one or more biomarker(s) in a sample from the cat, wherein the one or more biomarker(s) is selected from white blood cell count, haemoglobin, serum urea nitrogen, serum AST, serum chloride, serum total bilirubin, serum globulin, haematocrit, red blood cells count, serum albumin, and/or mean corpuscular hemoglobin concentration; and generating a report of the mortality risk and/or probability of a healthy lifespan of the cat.

The invention further provides a computer program product comprising computer implementable instructions for causing a programmable computer to determine the mortality risk and/or probability of a healthy lifespan of a cat given the level of one or more biomarker(s) from the cat, wherein the one or more biomarker(s) is selected from white blood cell count, haemoglobin, serum urea nitrogen, serum AST, serum chloride, serum total bilirubin, serum globulin, haematocrit, red blood cells count, serum albumin, and/or mean corpuscular hemoglobin concentration.

Advantageously, embodiments of the present invention in which levels of multiple biomarkers are determined enable the determination of mortality risk and/or probability of a healthy lifespan based on markers of multiple organ systems and functions. Accordingly, the use of multiple biomarkers advantageously encompassed a range of potential organ dysfunctions.

Evaluating the mortality risk or probability of survival for a cat allows one to test several aspects of the animal's wellbeing. First, it can predict whether this animal is more likely to need a dietary or supplement-based intervention. Second, it can be used to test the efficacy of a dietary or supplement-based intervention on aging. It also provides a specific set of biomarkers that can be tested to evaluate the mortality risk or survival probability of an animal and that are routinely tested in vet clinic or offices.

### DESCRIPTION OF DRAWINGS

FIG. 1—Identification of blood biomarkers predictive of mortality risk. A cox proportional hazard model was fit for each of the 28 biomarkers assessed, including sex. Values are adjusted for the p.value of each parameter to account for multiple comparison (by false discovery rate (fdr)). Parameters show are those with an adjusted fdr below 0.05.

FIG. 2—Demonstration of biomarkers that contribute to the predictive ability of the multi-parameter model for determining phenoage.

### DETAILED DESCRIPTION

Various preferred features and embodiments of the present invention will now be described by way of non-limiting examples. The skilled person will understand that they can combine all features of the invention disclosed herein without departing from the scope of the invention as disclosed.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Numeric ranges are inclusive of the numbers defining the range.

The publications discussed herein are provided solely for their disclosure prior to the filing date of the present application. Nothing herein is to be construed as an admission that such publications constitute prior art to the claims appended hereto.

The methods and systems disclosed herein can be used by veterinarians, health-care professionals, lab technicians, pet care providers and so on.

### Subject

The present methods are directed to feline subjects. Accordingly, the subject of the present invention is a cat. Preferably, the cat is a domestic cat. More preferably, the cat is a Domestic Shorthair cat.

### Sex

Suitably, the sex of the cat may be classified as male or female.

### Chronological Age

Chronological age may be defined as the amount of time that has passed from the subject's birth to the given date. Chronological age may be expressed in terms of years, months, days, etc.

Suitably, the present method may be applied to a cat of any chronological age. In certain embodiments, the cat may be at least about about 2 years old. Suitably, the cat may be at least about 2, at least about 3, at least about 4, at least about 5, at least about 6, at least about 7, at least about 8, at least about 9 or at least about 10 years old.

### Sample

The present invention comprises a step of determining the level of one or more biomarkers in one or more samples obtained from a subject.

Preferably, the sample is derived from blood. The sample may contain a blood fraction or may be whole blood. The sample preferably comprises blood plasma and/or serum. Techniques for collecting samples from a subject are well known in the art.

A suitable sample may be selected based on the biomarker(s) to be determined. By way of example, if the biomarkers are to be determined using a complete blood count (cbc), for example as part of a standard clinical complete blood count (cbc), a whole blood sample should be used.

The present method may be performed on one or more samples obtained from the subject. For example, the method may be performed using a first sample obtained at a given time point and a second sample obtained following a time interval after the first sample was obtained. The method may be performed more than once, on samples obtained from the same cat over a time period. For example, samples may be obtained repeatedly once per month, once a year, or once every two years. Suitably, the samples may be obtained around once per year (e.g. during an annual veterinary health check). This may be useful in determining the effects of a particular treatment or change in lifestyle—such as a dietary intervention or a change in exercise regime.

In one embodiment, the level of one or more biomarkers may be determined prior to a change in lifestyle (e.g. a dietary product intervention or a change in exercise regime). In another embodiment, the level of one or more biomarkers may be determined prior to, and after the e.g. dietary product intervention or change in exercise regime. The biomarker level may also be determined at predetermined times throughout the e.g. dietary product intervention or change in exercise regime. These predetermined times may be periodic throughout the e.g. dietary product intervention or change in exercise regime, e.g. every day or three days, or may depend on the subject being tested.

### Determining the Level of One or More Biomarkers in the Sample

The biomarkers used in the present invention can be determined using standard methods in the art and are typically measured as part of standard blood tests to determine the disease status of an animal. For example, the biomarkers are commonly determined as part of a standard clinical complete blood count (cbc) and standard clinical blood chemistry analysis.

A complete blood count provides information about blood cells and their properties; for example red blood cells, white blood cells, and platelets. An example complete blood count can comprise an automated process using flow cytometry or Coulter counter to determine cell number in the blood. In addition to determining cell count, such automated systems may also be capable of determining other blood biomarker readings depending on their complexity. Such systems are able to simultaneously measure blood cell counts as well as red blood cell volume, hemoglobin level, mean corpuscular hemoglobin level and hematocrit. For example, IDEXX Laboratories provide a hematology analyzer capable of determining white blood cell count (WBC), red blood cell count (RBC), platelet count (PLT), hemoglobin (HGB), hematocrit (HCT), mean red cell volume (MCV) and mean corpuscular hemoglobin (MCH) (IDEXX Laboratories Inc., ProCyte Dx Hematology Analyzer).

The levels of other biomarkers unrelated to blood cells, such as serum protein/enzyme and/or molecule biomarker levels, can be measured using chemical tests, in particular using automated chemistry analyser systems. These methods may utilize colorimetry-based approaches for quantification. For example, IDEXX Laboratories provide an automated chemistry analyzer able to quantify serum Albumin, serum Alkaline Phosphatase, serum Creatine Kinase, serum glucose, serum globulin, and serum Calcium (IDEXX Laboratories Inc., Catalyst One Chemistry Analyzer).

Accordingly, methods for determining the level of a biomarker used in the present invention may comprise assays that result in spectrophotometric changes (for example, chemical or antibody-linked changes that result in detectable signals at certain wavelengths). Such tests can be highly automated and efficient, and form the basis of many normal veterinary health check.

Suitably, the biomarker level may be determined after overnight fasting and measured using standard veterinary clinical practice.

The level of the individual biomarker species in the sample may be measured or determined by any suitable method known in the art. For example, mass spectrometry (MS), antibody detection methods, e.g. enzyme-linked immunoabsorbent assay (ELISA), non-antibody protein scaffolds (e.g. fibronectin scaffolds), radioimmuno-assay (RIA), or aptamers may be used. Other spectroscopic methods, chromatographic methods, labelling techniques, or quantitative chemical methods may also be used.

Suitable antibodies for use in methods described above are known in the art and/or may be generated using known techniques. Suitable test methods for detecting antibody levels include, but are not limited to, an immunoassay such as an enzyme-linked immunosorbent assay, radioimmunoassay, Western blotting and immunoprecipitation.

### Biomarkers

### White Blood Cell Count

White blood cells, also termed leukocytes, are a type of cell that are found in the blood. They have various immune-related functions, dependent on their sub-type: monocytes, lymphocytes, neutrophils, basophils and eosinophils. White blood cells contain a nucleus, and have a variable cell-shape that is also dependent on sub-type. White blood cell count is the number of this type of cell per volume of blood.

Methods of measuring white blood cell count, typically expressed in thousands of cells per microliter (10\hat{ }3/uL), are known in the art. White blood cell count measurements can be done manually on a blood smear using staining and microscopy techniques, but can also be carried out as part of an automated complete blood count (CBC). IDEXX Laboratories provide an automated hematology analyzer capable of white blood cell count measurements.

Suitably, increased white blood cell count may be associated with a negative effect on reducing mortality risk. Accordingly, increased white blood cell count may be associated an increased mortality risk.

### Haemoglobin

Hemoglobin is a transport protein in red blood cells. It consists of a tetramer of two alpha chains and two beta chains. Each peptide chain binds a heme group, which consists of a porphyrin ring with an iron ion bound. This group can reversibly bind oxygen which allows hemoglobin to function as an oxygen-transport carrier protein.

Methods of determining hemoglobin levels, typically expressed in grams per decilitre (g/dL), are well known in the art. The International Committee for Standardization in Haematology describe a standardized method utilising spectrophotometric determination of hemoglobin cyanide (Br J Haematol. 1967 April; 13:71-5) and this approach can be used in commercially available automated chemical analyzers such as that provided by IDEXX Laboratories.

Suitably, increased haemoglobin levels may be associated with a positive effect on reducing mortality risk. Accordingly, increased haemoglobin may be associated a reduced mortality risk.

### Serum Urea Nitrogen

Urea is a waste product from the breakdown of nitrogenous compounds, and is the main method by which nitrogen is excreted from the body. Urea is produced in the liver from the toxic by-product of protein metabolism, ammonia. Urea circulates in the blood, until it is removed by the kidneys and excreted in urine. Changes in the level of serum urea nitrogen, also known as blood urea nitrogen, which is a measure of the amount of urea in the blood, can be indicative of certain conditions and diseases. An overall rise in serum urea nitrogen can indicate kidney disease or starvation, whereas a fall in levels can be indicative of chronic or severe liver disease or low protein diet.

Methods of determining serum urea nitrogen, commonly expressed in milligrams per decilitre (mg/dL, natural log transformed), are well known in the art. The commonly termed Blood Urea Nitrogen (BUN) test is a common test as part of a chemistry panel performed on blood serum. In general, serum urea nitrogen can be determined by colorimetric or enzymatic conductivity methods, for example as described in EP0163367 and US2006/0046275. The automated chemistry analyzer available from IDEXX is a commercially available system capable of providing Serum Urea Nitrogen readings.

Suitably, increased serum urea nitrogen may be associated with a negative effect on reducing mortality risk. Accordingly, increased serum urea nitrogen may be associated an increased mortality risk.

### Serum AST

Aspartate aminotransferase, AST, is an enzyme that is involved in amino acid metabolism. AST is predominantly found in the liver and in muscle. Serum AST is a measure of the amount of this enzyme in the blood. High levels of AST in the blood can be indicative of liver or muscle damage.

Methods of determining serum AST, commonly expressed in units per litre (U/L, natural log transformed), are well known in the art. AST serum levels can be determined using colorimetric or electrochemical methods, such as those described in EP0523227 and WO2017/117231. The automated chemistry analyzer available from IDEXX (IDEXX Laboratories Inc., Catalyst One Chemistry Analyzer) is a commercially available system capable of providing Serum AST readings.

Suitably, increased serum AST may be associated with a negative effect on reducing mortality risk. Accordingly, increased serum AST may be associated an increased mortality risk.

### Serum Chloride

Serum Chloride is a measure of the concentration of chloride in the blood. Chloride is one of the most abundant electrolytes found in the blood, and is important for regulating the water content of the blood. Changes in the chloride levels in the blood can be indicative of fluctuations in total body water content, which can be caused by conditions such as vomiting or diarrhoea.

Methods of determining serum chloride, commonly expressed in millimoles per litre (mmol/L), are well known in the art. For example, colorimetric, coulometric, mercurimetric and chloride-specific ion electrode-based methods (Walker H K, Hall W D, Hurst J W, editors. Clinical Methods: The History, Physical, and Laboratory Examinations. 3rd edition. Boston: Butterworths; 1990. Chapter 197). Commercially available automated analyzers are capable of determining serum Chloride levels, such as described in Adin et al. (2020; Journal of veterinary internal medicine, 34(1), 53-64) or using an analyzer such as those that are commercially available from IDEXX laboratories.

Suitably, increased serum chloride levels may be associated with a positive effect on reducing mortality risk. Accordingly, increased serum chloride levels may be associated a reduced mortality risk.

### Serum Total Bilirubin

Serum Total Bilirubin is a measure of the amount of bilirubin in the blood. Bilirubin is a compound that is produced during the breakdown of heme from hemoglobin from old or damaged red blood cells or from breakdown of other heme containing proteins in other tissues, for example myoglobin. Bilirubin is processed by the liver, and products are excreted via bile and urine. Increased levels of serum total bilirubin can be indicative of impaired liver or bile duct function, or indicate an increased level of destruction of red blood cells. Increased levels of serum bilirubin can be observed by a yellowing of the skin, whites of the eyes and mucous membranes known as jaundice.

Methods of determining Serum Total Bilirubin, commonly expressed in milligrams per decilitre (mg/dL), are commonly known in the art. Bilirubin levels can be determined from blood samples via spectrophotometric, enzymatic, or, most commonly, diazo-dye colorimetric assays (described in Westwood A. (1991) The analysis of bilirubin in serum. Ann Clin Biochem 28; 119-130). Automatic analyzers are also commercially available from IDEXX for diagnostic use.

Suitably, increased Serum Total Bilirubin may be associated with a negative effect on reducing mortality risk. Accordingly, increased Serum Total Bilirubin may be associated an increased mortality risk.

### Serum Globulin

Serum globulin is a measure of the concentration of globular protein in the blood. Globular proteins are secreted mainly by the liver and a smaller proportion are secreted by immune cells. Albumin is the most abundant of the serum globulins. The remaining serum globulins can be separated into fractions based on their behaviour in electrophoresis separation methods. Immunoglobulins are an important part of the immune system and are secreted by immune cells. Examples of other serum globulins are immune system proteins such as complement, hormones and carrier proteins such as ferritin. Changes in the total level of serum globulin proteins can be indicative of certain conditions or diseases. An overall rise in serum globulins can indicate infection and an inflammatory immune response, whereas a fall in levels can be indicative of bleeding, gastrointestinal disease or severe malnutrition.

Methods of determining serum globulin levels, typically expressed in grams per decilitre (g/dL), are well known in the art. For example, chemical and physical methods are described in Tothova et al. (Veterinarni Medicina, 2016, 61: 475-496) and an automated chemistry analyzer available from IDEXX is also capable of measuring serum globulin levels.

Suitably, increased serum globulin levels may be associated with a negative effect on reducing mortality risk. Accordingly, increased serum globulin levels may be associated an increased mortality risk.

### Hematocrit

Hematocrit is the percentage by volume of red blood cells in the blood. Hematocrit levels that fall outside normal values can be indicative of diseases or conditions that result in a greater or lesser proportion of red blood cells in the blood than normal. A high hematocrit can be indicative of conditions such as dehydration, whilst a low hematocrit can be indicative of an anemia, due to bleeding, hemolysis or decreased production of red blood cells.

Methods of determining haematocrit, typically expressed as a percentage of blood volume (%), are well known in the art. Measurement can be carried out manually using packed cell volume by centrifuging blood in a microhematocrit tube. Alternatively, haematocrit can be calculated from mean red cell volume and the red blood cell count, both of which can be measured directly by modern hematology analyzers in a standard complete blood count (CBC).

Suitably, increased haematocrit levels may be associated with a positive effect on reducing mortality risk. Accordingly, increased haematocrit may be associated a reduced mortality risk.

### Red Blood Cell Count

Red blood cells, also known as red blood corpuscles, are the most abundant cells present in the blood. These cells do not contain a nucleus, and instead consist mainly of hemoglobin contained within the cell membrane to maximise their oxygen-carrying potential. Red blood cell counts that are above or below normal levels are indicative of disorders or disease. Low red blood count can indicate hemolysis, blood loss, or reduced production of red blood cells that can result from multiple causes. High red blood cell count can indicate a relative increase of red blood cells per volume of blood compared to normal, due to dehydration or increased red blood cell production.

Methods of measuring red blood cell count, typically expressed in thousands of cells per microliter (10\hat{ }3/uL), are well known in the art. Red blood cell count measurements can be done manually on a blood smear using microscopy, but are commonly carried out as part of an automated complete blood count (CBC).

Suitably, increased red blood cell count may be associated with a positive effect on reducing mortality risk. Accordingly, increased red blood cell count may be associated a reduced mortality risk.

### Serum Albumin

Serum Albumin is a globular protein found in the blood. It is a 65 kDa protein comprised of three homologous domains. Albumin has an important role in the blood, where it regulates oncotic pressure, preventing loss of fluid from the blood to the tissues, and acting as a transport protein for fatty acids, bilirubin, heme, heavy metals, hormones and certain drugs. Albumin is highly abundant in the blood, accounting for 25-50% of total plasma protein by weight, and is produced by the liver. Abnormally high or low levels of Albumin in the blood can be indicative of liver or kidney disease.

Methods of measuring serum Albumin levels, typically expressed in grams per decilitre (g/dL), are well known in the art, and include zone electrophoresis, dye-binding assays involving bromocresol green (BCG) or bromocresol purple (BCP), and ELISA methods. For example, Eagle Biosciences offer an ELISA-based assay for canine serum albumin (Eagle Biosciences Inc., product code SKU: CAE49-K01). Stokol et al. describe the use of automated systems using a BCG-binding assay in determining serum Albumin levels in cats in a clinical setting (Vet Clin Pathol. 2001; 30(4):170-176). Additionally, IDEXX Laboratories offer an automated chemistry analyzer that can conduct measurement of serum Albumin levels as part of a combined blood test.

Suitably, increased serum albumin levels count may be associated with a positive effect on reducing mortality risk. Accordingly, increased serum albumin levels may be associated a reduced mortality risk.

### Mean Corpuscular Hemoglobin Concentration

Mean corpuscular hemoglobin concentration (MCHC) is the average mass of haemoglobin per given volume of packed red blood cell. MCHC values that are outside normal ranges can be indicative of certain diseases such as hypochromic anemias.

Methods of measuring MCHC, typically expressed in grams per decilitre (g/dL), are well known in the art. Methods commonly comprise calculation from observed values of hemoglobin level and hematocrit that can be measured during a complete blood count (CBC). When a CBC is carried out using a hematology analyzer as described above, the MCHC is calculated using the Hemoglobin concentration, Red Blood Cell count (RBC) and Mean Corpuscular Volume (MCV).

Suitably, increased MCHC may be associated with a positive effect on reducing mortality risk. Accordingly, increased MCHC may be associated a reduced mortality risk.

### Serum Sodium

Serum Sodium is a measure of the concentration of sodium in the blood. Sodium is the most abundant cation in the blood, and functions to maintain fluid distribution and osmotic pressure in the blood. Changes in serum sodium levels can be indicative of kidney disease, or as a result of severe diarrhea or vomiting.

Methods of measuring serum sodium levels, typically expressed in millimoles per litre (mmol/L), are well known in the art. Most modern tests use a direct or indirect ion-selective electrode method. Automated analyzers are also commercially available that are able to carry out this measurement on a blood sample such as those available from IDEXX.

Suitably, increased serum sodium levels may be associated with a negative effect on reducing mortality risk. Accordingly, increased serum sodium levels may be associated an increased mortality risk.

### Serum Cholesterol

Serum Cholesterol is a measure of the amount of cholesterol present in the blood. Cholesterol is an essential sterol that forms part of cellular membranes and is involved in signalling and is a metabolic precursor for multiple steroid hormones. The cholesterol in blood is transported via lipoproteins, which are particles containing amphipathic proteins and lipids. Changes in the level of cholesterol in the blood can be indicative some conditions or diseases. Low cholesterol can be indicative of liver disease, severe small intestinal disease, or a low fat diet.

Methods of measuring serum cholesterol, commonly expressed in milligrams per decilitre (mg/dL), are well known in the art. These include classical chemical methods such as the Abell-Kendall method, fluorometric-enzymatic assays commonly used in automated systems, and mass spectrometry-based methods, all of which are discussed in Li et al. (2019; J Food Drug Anal.; 27(2):375-386). Commercial automated analyzers capable of measuring serum cholesterol levels are available such as those from IDEXX.

Suitably, increased serum cholesterol may be associated with a negative effect on reducing mortality risk. Accordingly, increased serum cholesterol may be associated an increased mortality risk.

### Serum Potassium

Serum Potassium is a measure of the concentration of potassium in the blood. Potassium is an important electrolyte that is predominantly found intracellularly, and the gradient of potassium across the extracellular membrane is responsible for regulating fluid balance, as well as generating action potentials essential for nerve and muscle function. The proportion of potassium found in the extracellular fluid is low, but serum concentrations can indicate certain diseases and conditions. Low serum potassium can be indicative of chronic kidney disease, diarrhea and vomiting, or a diet deficient in potassium, and can result in muscle weakness. High serum potassium can be indicative of kidney disease or hormonal disorders.

Methods of measuring serum potassium levels, commonly expressed in millimoles per litre (mmol/L), are well known in the art. Potassium levels in serum can be determined by the use of a colorimetric method or a direct or indirect ion-selective electrode method. A commercial automated analyzer is available from IDEXX that is capable of measuring serum Potassium levels.

Suitably, increased serum potassium levels may be associated with a negative effect on reducing mortality risk. Accordingly, increased serum potassium levels may be associated an increased mortality risk.

### Serum Alkaline Phosphatase

Alkaline Phosphatase (ALP) is an enzyme that has an important role in liver metabolism and in skeletal development. It is an 86 kDa homodimeric protein. High levels of this protein in the blood can be indicative of liver damage or bone disease.

Methods of measuring serum alkaline phosphatase levels, typically expressed as units per litre (U/L, natural log transformed), are well known in the art, and most often consist of quantification of enzymatic activity via colorimetric assay. An automated chemistry analyser is available from IDEXX Laboratories that is capable of measuring serum alkaline phosphatase levels.

Suitably, increased serum alkaline phosphatase levels may be associated with a positive effect on reducing mortality risk. Accordingly, increased serum alkaline phosphatase levels may be associated a reduced mortality risk.

### Serum GGT

Gamma-glutamyl transferase (GGT) is an enzyme that is involved in glutathione metabolism and is mainly found in the liver. Serum GGT is a measure of the amount of GGT in the blood. Increased levels of GGT in the blood may be indicative of liver disease, such as cholestasis.

Methods of measuring serum GGT levels, typically expressed as grams per decilitre (g/dL, natural log transformed), are well known in the art. A colorimetric enzymatic assay can be used to determine GGT levels, and commercial automated analyzers are available for this purpose such as those available from IDEXX.

Suitably, increased serum GGT levels may be associated with a negative effect on reducing mortality risk. Accordingly, increased serum GGT levels may be associated an increased mortality risk.

### Combinations of Biomarkers

Whilst individual biomarkers may have predictive value in the methods of the present invention, the quality and/or the predictive power of the methods may be improved by combining values from multiple biomarkers.

Thus the present method may involve determining the level of at least two biomarkers from those described herein. For instance, the method may comprise determining the level of two or more biomarkers selected from white blood cell count, haemoglobin, serum urea nitrogen, serum AST, serum chloride, serum total bilirubin, serum globulin, haematocrit, red blood cells count, serum albumin, mean corpuscular hemoglobin concentration, serum sodium, serum cholesterol, serum potassium, serum alkaline phosphatase, and/or serum GGT in one or more samples.

The term “one or more biomarkers” as used herein may include at least one, at least two, at least three, at least four, at least five, at least six, at least seven, at least eight, at least nine, at least ten, at least eleven, at least twelve or at least thirteen biomarkers.

The term “one or more biomarkers” as used herein may include one, two, three, four, five, six, seven, eight, nine, ten, eleven, twelve or thirteen biomarkers.

Suitably, the present method may comprise determining the level of white blood cell count, serum haemoglobin, serum urea nitrogen and serum AST in one or more samples. Advantageously, this combination of four biomarkers has been determined to provide a notable prediction of mortality risk and/or probability of a healthy lifespan. The predictive ability may be further increased by incorporating one or more of the additional biomarkers selected from serum chloride, serum total bilirubin, serum globulin, haematocrit, red blood cells count, serum albumin, mean corpuscular hemoglobin concentration, serum sodium, serum cholesterol, serum potassium, serum alkaline phosphatase, and serum GGT.

Suitably, the present method may comprising determining the level of each of white blood cell count, haemoglobin, serum urea nitrogen, serum AST, serum chloride, serum total bilirubin, serum globulin, red blood cell count, serum sodium, serum cholesterol, serum potassium, serum alkaline phosphatase, and serum GGT in one or more samples.

### Comparison to a Reference or Control

The present method may further comprise a step of comparing the level of the individual biomarker(s) in the test sample to one or more reference or control values. The reference value may be associated with a pre-defined mortality risk and/or probability of a healthy lifespan. In some embodiments, the reference value is a value obtained previously for a subject or group of subjects with a known mortality outcome. The reference value may be based on an average level, e.g. a mean or median level, from a group of subjects with known chronological age, breed, sex and/or mortality outcome. Preferably, the reference value may be based on an average level, e.g. a mean or median level, from a group of subjects with known chronological age, breed, sex and mortality outcome.

Combining the Biomarker Levels with Further Measures and/or Characteristics

Suitably, the present method further comprises combining the level of the one or more biomarkers with one or more of the chronological age and/or sex of the cat. By combining this information, an improved model is provided for the mortality risk of the cat and/or probability of a healthy lifespan.

In a preferred embodiment, levels of one or biomarkers as defined herein are determined for a sample from the cat and these levels are combined with the chronological age of the cat in order to determine a mortality risk and/or probability of a healthy lifespan for the cat.

Preferably, the mortality risk and/or probability of a healthy lifespan is represented as a phenotypic age (Phenoage), which is given by the following formula:

$$Phenoage = ln ( \frac{{γ}_{2} * {e}^{xb} * \{ {e}^{γ * age} - 1 \}}{{e}^{{β}_{02}} * γ} + 1 ) * \frac{1}{{γ}_{2}}$$

where xb is the sum of the value of each biomarker(s), multiplied by their respective coefficients according to formula (2):

$$xb = {\sum }_{u = 1}^{p} {x}_{u} {β}_{u} + {β}_{0}$$

and
c. using the phenotypic age to determine a mortality risk and/or probability of a healthy lifespan for the cat.

The coefficient value for each parameter typically depends on the measurement units of all the variables in the model. As would be understood by the skilled person, the exact value for each coefficient value will therefore depend on, for example, the number and nature of the different parameters used in the model and the nature of the training data provided (e.g. the breed(s) of the cats in the training data set). Accordingly, routine statistical methods may be applied to a training data set in order to arrive at coefficient values for use in above formula. Such methods include, for example, computation of two gompertz functions on a training set (e.g. where the status of the cat (alive or dead) is known), one that models survival as a function of the selected biomarkers, chronological age, and sex (model 1) and a second function that only considers chronological age, and sex (model 2). These models may be fit using the flexsurv package (v 2.1) in the R software environment.

Suitably, a negative coefficient for a given biomarker means that a higher level of the biomarker has a positive effect on reducing mortality risk and/or a lower level of the biomarker has a negative effect on reducing mortality risk. Suitably, a positive coefficient for a given biomarker means that a higher level of the biomarker has a negative effect on reducing mortality risk and/or a lower level of the biomarker has a positive effect on reducing mortality risk.

The phenotypic age may be defined as the time variable (“chronological age”) at which the survival probability of the animal given by model 2 is equal to the survival probability at their chronological age given by the model 1.

The phenotypic age (i.e. phenoage) of the cat may be expressed in terms of years, months, days, etc.

Preferably, the mortality risk and/or probability of a healthy lifespan is represented as the difference between phenoage and chronological age of the cat. This difference may be referred to as the phenoage advance of the cat.

For example, an increase in phenoage compared to chronological age may be indicative of an increased mortality risk for the cat. For example, a decrease in phenoage compared to chronological age may be indicative of a decreased mortality risk for the cat. As an illustration, the present inventors determined that the difference between phenoage and chronological age (phenoage advance) was associated with a significant increase in mortality risk, and the magnitude of the effect was calculated to be a hazard ratio of 1.36 for a 1 year increase in phenoage compared to chronological age (see Example 3). In other words, the inventors determined that a 1 year increase in phenoage vs chronological age was associated with a risk of mortality approximately 35% higher at any given point in life.

### Subject Stratification

The mortality risk and/or probability of a healthy lifespan determined by the method of the present invention may also be compared to one or more pre-determined thresholds. Using such thresholds, subjects may be stratified into categories which are indicative of determined mortality risk, e.g. low, medium or high determined mortality risk and/or probability of a healthy lifespan. The extent of the divergence from the thresholds is useful to determine which subjects would benefit most from certain interventions. In this way, dietary intervention and modification of lifestyle can be optimised. The determined mortality risk and/or probability of a healthy lifespan may be presented in terms of a numerical score or percentage, which—for example—may be indicative of determined mortality risk and/or probability of a healthy lifespan compared to a control or reference population.

### Method for Selecting/Monitoring a Lifestyle or Dietary Regime of a Subject

In a further aspect, the present invention provides a method for selecting a lifestyle or dietary regime for a subject. The modification in lifestyle may be any change as described herein, e.g. a dietary intervention and/or a change in exercise regime. The modification in lifestyle may be administration of a therapeutic modality.

The lifestyle or dietary regime may be applied to the cat for any suitable period of time. After said period of time, the cat's mortality risk and/or probability of a healthy lifespan may be determined again using the present method in order to determine the efficacy of the lifestyle or dietary regime for reducing the mortality risk and/or increasing the probability of a healthy lifespan of the cat. By way of example, the lifestyle or dietary regime may be applied for at least 2, at least 4, at least 8, at least 16, at least 32, or at least 64 weeks. The lifestyle or dietary regime may be applied for at least 3, at least 6, at least 12, at least 24, at least 36, at least 48 or at least 60 months.

Preferably the modification is a dietary intervention as described herein. By the term “dietary intervention” it is meant an external factor applied to a subject which causes a change in the subject's diet. More preferably the dietary intervention includes the administration of at least dietary product or dietary regimen or a nutritional supplement.

The dietary intervention may be a meal, a regime of meals, a supplement or a regime of supplements.

The dietary intervention or dietary product described herein may be any suitable dietary regime, for example, a calorie-restricted diet, a senior diet, a low protein diet, a phosphorous diet, low protein diet, potassium supplement diet, polyunsaturated fatty acids (PUFA) supplement diet, anti-oxidant supplement diet, a vitamin B supplement diet, liquid diet, selenium supplement diet, omega 3-6 ratio diet, or diets supplemented with carnitine, branched chain amino acids or derivatives, nucleotides, nicotinamide precursors such as nicotinamide mononucleotide (MNM) or nicotinamide riboside (NR) or any combination of the above.

Suitably, the dietary intervention or dietary product may be a calorie-restricted diet, a senior diet or a low protein diet. Suitably, the dietary intervention or dietary product may be a calorie-restricted diet. Suitably, the dietary intervention or dietary product may be a low protein diet.

A dietary intervention may be determined based on the baseline maintenance energy requirement (MER) of the cat. Suitably, the MER may be the amount of food that stabilizes the cat's body weight (less than 5% change over three weeks).

By way of example, it is generally understood that younger, growing cats benefit from a high energy/high protein diet; however, older cats may have a lower energy requirement and therefore diets can be appropriately modified. In particular, many manufacturers produce a ‘senior’ range of cat food which is lower in calories, higher in fibre but has suitable levels of protein and fat for an older cat.

Suitably, a calorie-restricted diet may comprise about 60%, about 65%, about 75% or about 80% of the cat's MER. Suitably, a calorie-restricted diet may comprise about 60% or about 75% of the cat's MER.

Suitably, a low-protein diet may comprise less than 20% protein (% dry matter). For example, a low-protein diet may comprise less than 15% or less than 10% protein (% dry matter).

These diets are generally recommended based upon the chronological age of a cat. For example, it may be recommended that a cat is switched to a senior diet around 7 or 8 years old. However, in the context of the present invention, the determination of an increased mortality risk and/or reduced probability of a healthy lifespan for a cat compared to what would be expected given its chronological age may allow a determination to switch the cat to a senior diet at an earlier age. In contrast, a cat with a reduced mortality risk and/or increased probability of a healthy lifespan compared to its chronological age may be able to stay on a high energy/high protein diet for longer.

The dietary intervention may comprise a food, supplement and/or drink that comprises a nutrient and/or bioactive that mimics the benefits of caloric restriction (CR) without limiting daily caloric intake. For example, the food, supplement and/or drink may comprise a functional ingredient(s) having CR-like benefits. Suitably, the food, supplement and/or drink may comprise an autophagy inducer. Suitably, the food, supplement and/or drink may comprise fruit and/or nuts. Suitable examples include, but are not limited to, pomegranate, strawberries, blackberries, camu-camu, walnuts, chestnuts, pistachios, pecans. Suitably, the food, supplement and/or drink may comprise probiotics with or without fruit extracts or nut extracts.

Modifying a lifestyle of the subject also includes indicating a need for the subject to change lifestyle, e.g. prescribing more exercise. Similar to a dietary intervention, the determination of an increased mortality risk and/or reduced probability of a healthy lifespan for a cat compared to what would be expected given its chronological age may allow a determination a switch the cat to an appropriate exercise regime.

Modifying a lifestyle of the subject also includes recommending a therapeutic modality or regimen. The therapeutic modality or regimen may be a modality useful in treating and/or preventing—for example—arthritis, dental diseases, endocrine disorders, heart disease, diabetes, liver disease, kidney disease, prostate disorders, cancer and behavioural or cognitive disorders. Suitably, prophylactic therapies may be administered to a cat identified as being at risk of such disorders due to increase mortality risk (phenoage) and/or on the basis of particular biomarkers which are known to be associated with disease-relevant pathways. In other embodiments, cats determined to be at risk of certain conditions (due to increase mortality risk (phenoage) and/or on the basis of particular biomarkers which are known to be associated with disease-relevant pathways) may be monitored more regularly so that diagnosis and treatment can begin as early as possible.

The present invention may thus advantageously enable the identification of cats that are expected to respond particularly well to a given intervention (e.g. lifestyle or dietary regime). The intervention can thus be applied in a more targeted manner to cats that are expected to respond.

The invention further provides a method for determining the efficacy of a lifestyle or dietary regime for reducing the mortality risk and/or increasing the probability of a healthy lifespan for a cat, said method comprising:
a. determining a first mortality risk and/or probability of a healthy lifespan for the cat according to the method of the first aspect of the invention;
b. applying a lifestyle or dietary regime to the cat;
c. after a time period of applying the lifestyle or dietary regime to the cat, determining a second mortality risk and/or probability of a healthy lifespan for the cat according to the method of the first aspect of the invention;
d. determining if there has been a change in the first and second mortality risk and/or probability of a healthy lifespan for the cat after the time period of following the lifestyle or dietary regime.

The invention also provides a method for determining the efficacy of a lifestyle or dietary regime for reducing the mortality risk and/or increasing the probability of a healthy lifespan determined for a cat, said method comprising:
a. applying a lifestyle or dietary regime to the cat, wherein the lifestyle or dietary regime has been selected according to a method comprising performing the method according to the first aspect of the invention; and selecting a suitable lifestyle or dietary regime based on the mortality risk and/or probability of a healthy lifespan determined;
b. determining a second mortality risk and/or probability of a healthy lifespan for the cat by performing the method of the first aspect of the invention after a time period of applying the lifestyle or dietary regime to the cat;
c. determining if there has been a change in mortality risk and/or probability of a healthy lifespan for the cat after the time period of following the lifestyle or dietary regime.

The invention further provides a method for determining the efficacy of a lifestyle or dietary regime for reducing the mortality risk and/or increasing the probability of a healthy lifespan of a cat, said method comprising:
a. selecting a lifestyle or dietary regime for the cat according to a method comprising performing the method according to the first aspect of the invention; and selecting a suitable lifestyle or dietary regime based on the mortality risk determined;
b. applying the lifestyle or dietary regime to the cat;
c. after a time period of applying the lifestyle or dietary regime to the cat, determining a second mortality risk and/or probability of a healthy lifespan for the cat according to the method of the first aspect of the invention;
d. determining if there has been a change in mortality risk and/or probability of a healthy lifespan for the cat between step a. and step c.

A reduction in the second (or subsequent) mortality risk determined for the cat compared to the first (or earlier) mortality risk and/or probability of a healthy lifespan determined for the cat after a period of applying the lifestyle or dietary regime is indicative that the lifestyle or dietary regime is effective in reducing the mortality risk and/or probability of a healthy lifespan for the cat.

The mortality risk and/or probability of a healthy lifespan for the cat may be determined prior to and after the lifestyle or dietary regime has been applied to the cat. The mortality risk and/or probability of a healthy lifespan for the cat may also be determined at predetermined times throughout the application of the lifestyle or dietary regime. These predetermined times may be periodic throughout the lifestyle or dietary regime, e.g. every day or three days, every week, every two weeks, every month, every two months, every 6 months, every year or every two years. The predetermined times may depend on the subject being tested. Suitably, the lifestyle or dietary regime may have been applied to the cat for a period before the first mortality risk is determined; however, the effectiveness of the lifestyle or dietary regime for reducing mortality risk may still be monitored by determining a mortality risk at two or more predetermined times during the application of the lifestyle or dietary regime.

The invention further provides a method for determining the efficacy of a lifestyle or dietary regime for reducing the mortality risk and/or increasing the probability of a healthy lifespan for a cat, said method comprising:
a. determining a first mortality risk and/or probability of a healthy lifespan for the cat according to the method of the first aspect of the invention;
b. applying a lifestyle or dietary regime to the cat;
c. after a time period of applying the lifestyle or dietary regime to the cat, determining a second mortality risk and/or probability of a healthy lifespan for the cat according to the method of the first aspect of the invention;
d. determining if there has been a change in the first and second mortality risk and/or probability of a healthy lifespan for the cat after the time period of following the lifestyle or dietary regime.

The invention also provides a method for determining the efficacy of a lifestyle or dietary regime for reducing the mortality risk and/or increasing the probability of a healthy lifespan determined for a cat, said method comprising:
a. applying a lifestyle or dietary regime to the cat, wherein the lifestyle or dietary regime has been selected according to a method comprising performing the method according to the first aspect of the invention; and selecting a suitable lifestyle or dietary regime based on the mortality risk determined;
b. determining a second mortality risk and/or probability of a healthy lifespan for the cat by performing the method of the first aspect of the invention after a time period of applying the lifestyle or dietary regime to the cat;
c. determining if there has been a change in mortality risk and/or probability of a healthy lifespan for the cat after the time period of following the lifestyle or dietary regime.

The invention further provides a method for determining the efficacy of a lifestyle or dietary regime for reducing the mortality risk and/or increasing the probability of a healthy lifespan of a cat, said method comprising:
a. selecting a lifestyle or dietary regime for the cat according to a method comprising performing the method according to the first aspect of the invention; and selecting a suitable lifestyle or dietary regime based on the mortality risk determined;
b. applying the lifestyle or dietary regime to the cat;
c. after a time period of applying the lifestyle or dietary regime to the cat, determining a second mortality risk and/or probability of a healthy lifespan for the cat according to the method of the first aspect of the invention;
d. determining if there has been a change in mortality risk for the cat between step a. and step c.

A reduction in the second (or subsequent) mortality risk determined for the cat compared to the first (or earlier) mortality risk determined for the cat after a period of applying the lifestyle or dietary regime is indicative that the lifestyle or dietary regime is effective in reducing the mortality risk for the cat.

The mortality risk and/or probability of a healthy lifespan for the cat may be determined prior to and after the lifestyle or dietary regime has been applied to the cat. The mortality risk and/or probability of a healthy lifespan for the cat may also be determined at predetermined times throughout the application of the lifestyle or dietary regime. These predetermined times may be periodic throughout the lifestyle or dietary regime, e.g. every day or three days, every week, every two weeks, every month, every two months, every 6 months, every year or every two years. The predetermined times may depend on the subject being tested. Suitably, the lifestyle or dietary regime may have been applied to the cat for a period before the first mortality risk and/or probability of a healthy lifespan is determined; however, the effectiveness of the lifestyle or dietary regime for reducing mortality risk and/or probability of a healthy lifespan may still be monitored by determining a mortality risk and/or probability of a healthy lifespan at two or more predetermined times during the application of the lifestyle or dietary regime.

### Use of a Dietary Intervention

In one aspect, the present invention provides a dietary intervention for use in reducing the mortality risk and/or increasing the probability of a healthy lifespan of a cat, wherein the dietary intervention is administered to a cat with a mortality risk and/or probability of a healthy lifespan determined by the present method.

In another aspect, the present invention provides the use of a dietary intervention to reduce the predicted mortality risk and/or increase the probability of a healthy lifespan of a cat, wherein the dietary intervention is administered to a cat with a mortality risk and/or probability of a healthy lifespan determined by the present method.

As described herein, the dietary intervention may be a dietary product or dietary regimen or a nutritional supplement.

### Computer Program Product

The present methods may be performed using a computer. Accordingly, the present methods may be performed in silico.

The methods described herein may be implemented as a computer program running on general purpose hardware, such as one or more computer processors. In some embodiments, the functionality described herein may be implemented by a device such as a smartphone, a tablet terminal or a personal computer.

In one aspect, the present invention provides a computer program product comprising computer implementable instructions for causing a programmable computer to determine the mortality risk and/or probability of a healthy lifespan of a cat as described herein.

In another aspect, the present invention provides a computer program product comprising computer implementable instructions for causing a device to determine the mortality risk and/or probability of a healthy lifespan of a dig given the levels of one or more biomarkers from the user, wherein the biomarkers are selected from the one or more biomarkers as defined herein. Preferably, the biomarker levels are fasting levels. The computer program product may also be given additional parameters or characteristics for the cat. As described herein, the additional parameters or characteristics may include chronological age, breed and sex.

In one embodiment, the user inputs into the device levels of one or more of the biomarkers as defined herein, optionally along with chronological age, breed and sex. The device then processes this information and provides a determination of a mortality risk and/or probability of a healthy lifespan for the cat.

The device may generally be a server on a network. However, any device may be used as long as it can process biomarker data and/or additional parameters or characteristic data using a processor, a central processing unit (CPU) or the like. The device may, for example, be a smartphone, a tablet terminal or a personal computer and output information indicating the determined mortality risk and/or probability of a healthy lifespan for the cat. The determined mortality risk and/or probability of a healthy lifespan may be presented in terms of a numerical score or percentage, which—for example—may be indicative of determined mortality risk and/or probability of a healthy lifespan compared to a control or reference population.

Those skilled in the art will understand that they can freely combine all features of the present invention described herein, without departing from the scope of the invention as disclosed.

### EXAMPLES

The invention will now be further described by way of examples, which are meant to serve to assist the skilled person in carrying out the invention and are not intended in any way to limit the scope of the invention.

### Example 1—Determination of Blood Biomarkers Associated with Mortality Risk in Cats

Predictive blood biomarkers were determined from a biomarker panel consisting of a standard clinical complete blood count (cbc) and standard clinical blood chemistry analysis. Serum samples were taken after overnight fasting and measured using standard veterinary clinical practice.

TABLE 1
Clinical complete blood count (cbc)
and clinical blood chemistry analysis

| Parameter name | Unit of measure |
|---|---|
| Hematocrit | % |
| Hemoglobin | g/dL |
| Mean Corpuscular Hemoglobin | pg |
| Mean Corpuscular Hemoglobin concentration | g/dL |
| Mean Red Cell Volume | fL |
| Platelet | 10\hat{ }3/uL |
| Red blood cells | 10\hat{ }3/uL |
| White blood cells | 10\hat{ }3/uL |
| Serum Albumin Plus | g/dL |
| Serum Alkaline Phosphatase * | U/L |
| Serum ALT * | U/L |
| Serum AST * | U/L |
| Serum Calcium | mg/dL |
| Serum Chloride | mmol/L |
| Serum Cholesterol | mg/dL |
| Serum Cretaine Kinase * | IU/L |
| Serum Creatinine, Jaffe Method * | mg/dL |
| Serum GGT * | g/dL |
| Serum Globulin | g/dL |
| Serum Glucose | mg/dL |
| Serum Magnesium | mg/dL |
| Serum Phosphorus | mg/dL |
| Serum Potassium | mmol/L |
| Serum Sodium | mmol/L |
| Serum Total Bilirubin * | mg/dL |
| Serum Total Protein | g/dL |
| Serum Triglycerides * | mg/dL |
| Serum Urea Nitrogen * | mg/dL |

* value were log-transformed using natural logarithm.

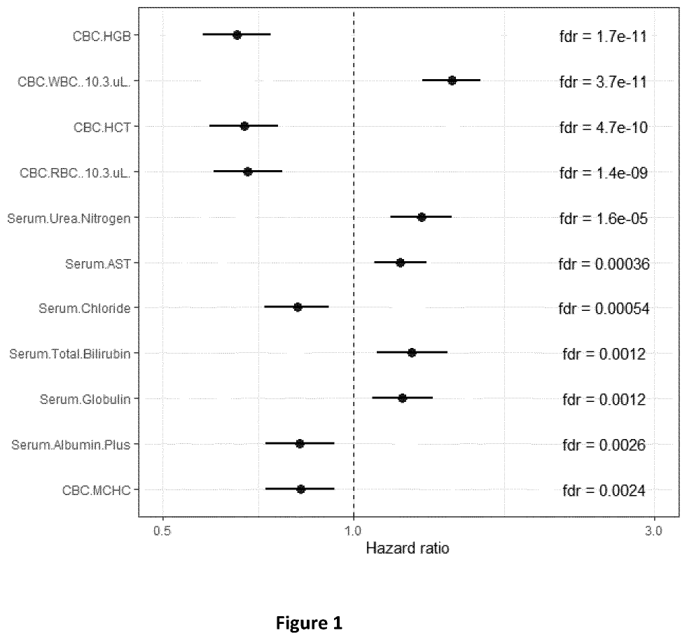

We used a longitudinal study of cats for which we have repeated measurement of these parameters as well as information about the status of the cat (alive or dead) and their sex. The analysis was directed to the Domestic Shorthair breed. Then we organize the data using the R programming language. For each cat, we record the biomarkers as time dependent covariates using time intervals open on the left and closed on the right (i.e. (tstart, tstop]), where the biomarker information corresponds to the start of the interval and the event (alive or dead) is recorded as the last tstop value. For this purpose, we use the tmerge function of the survival package in R (v. 3.2-13). Then, we fit a cox proportional hazard model to this data individually for each of the 28 biomarkers and sex as covariates. We then adjust the p.value of each parameter to account for multiple comparison (by false discovery rate (fdr)) and selected features with an adjusted fdr below 0.05 (FIG. 1).

Using this method, we identified 11 biomarkers that are individually predictive of the survival probability in cats:
Hemoglobin (g/dL)
White blood cells count (10\hat{ }3 per ul)
Hematocrit (%)
Red blood cells count (10\hat{ }3 per ul)
Serum Urea Nitrogen (mg/dL, natural log transformed)
Serum AST (U/L, natural log transformed)
Serum Chloride (mmol/L)
Serum Total Bilirubin (mg/dL)
Serum Globulin (g/dL)
Serum Albumin (g/dL)
Mean Corpuscular Hemoglobin concentration (g/dL)

### Example 2—Multi-Parameter Model for Predicting Mortality Risk

Next, we constructed the best model that would consider multiple parameters simultaneously, as this is more likely to cover a wide range of organ dysfunctions that occur with age. However, selecting several features that might be correlated with each other is subject to bias. To avoid this issue, we used a penalized regression method using the glmnet package (v4.1-3). We fit a LASSO-penalized cox proportional hazard model on data and used 20-fold cross validation to compare different values of the penalization parameter lambda. This approach leads to the selection of the top 13 most predictive blood biomarkers for survival, by order of importance as shown below:
White blood cells count (10\hat{ }3 per ul)
Hemoglobin (g/dL)
Serum Urea Nitrogen (mg/dL, natural log transformed)
Serum AST (U/L, natural log transformed)
Serum Chloride (mmol/L)
Serum Total Bilirubin (mg/dL)
Serum Globulin (g/dL)
Serum Sodium (mmol/L)
Serum Cholesterol (md/dL)
Serum Potassium (mmol/L)
Serum Alkaline Phosphatase (U/L)
Serum GGT (g/dL)
Red blood cells count (10\hat{ }3 per ul)

To extract the phenotypic age of the animal, we compute two different gompertz functions on our training set, one that models survival as a function of the selected biomarkers and age (model 1) and a second function that only considers age (model 2). These models are fit using the flexsurv package (v 2.1) The phenotypic age is defined as the time variable (“age”) at which the survival probability of the animal given by model 2 is equal to the survival probability at their chronological age given by the model 1. This leads to a mathematical function connecting the blood biomarkers to the phenoage and is given by the following formula:

$$Phenoage = ln ( \frac{{γ}_{2} * {e}^{xb} * \{ {e}^{γ * age} - 1 \}}{{e}^{{β}_{02}} * γ} + 1 ) * \frac{1}{{γ}_{2}}$$

Where xb is the sum of the value of each biomarkers multiplied by their respective coefficients. The coefficients are given by the two gompertz function trained on our training sets.

As an example, the coefficients, as well as the γ and γ₂ values have been measured from our training set for the complete list of biomarkers and are given in Table 1.

$$xb = {\sum }_{u = 1}^{p} {x}_{u} {β}_{u} + {β}_{0}$$

TABLE 2
Coefficients and γ values have been measured from training set

| | Coefficient |
|---|---|
| γ | 0.1968988 |
| β₀ | −2.943895 |
| β White blood cells count | 0.03858239 |
| β Hemoglobin | −0.1590607 |
| β Red blood cells count | −0.1234486 |
| β Serum Cholesterol | 0.002024294 |
| β Serum Potassium | 0.1692257 |
| β Serum Chloride | −0.08637897 |
| β Serum Sodium | 0.04714968 |
| β Serum Urea Nitrogen | 0.3992681 |
| β Serum Total Bilirubin | 2.867167 |
| β Serum AST | 0.4198243 |
| β Serum Alkaline Phosphatase | −0.3322447 |
| β Serum Globulin | 0.1615517 |
| β Serum GGT | 0.07676414 |
| γ₂ | 0.2937868 |
| β₀₂ | −5.7844426 |

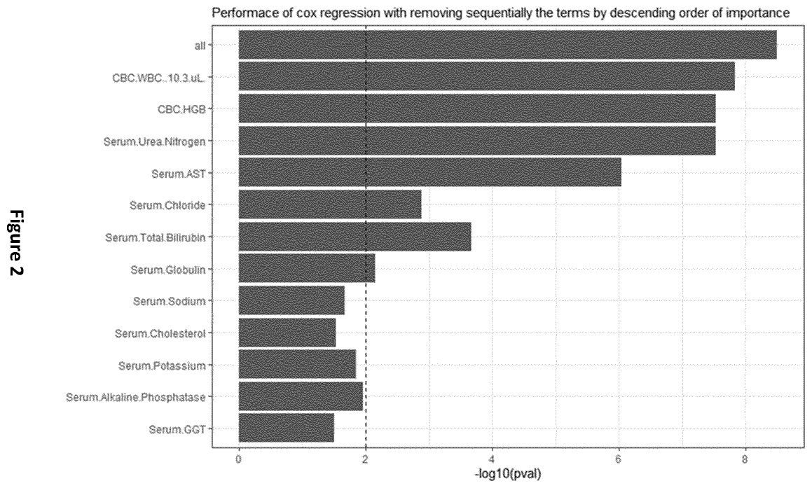

Further, by reducing the set of 13 biomarkers by systematically removing one biomarker, starting for the top of the list, we observed a reduction in the strength of the survival prediction (p value). The drop was most pronounced with the first four parameters, confirming their biggest contribution, but we observed a change in quality of prediction by each reduction of the set, showing that each parameter contributes to the overall prediction (FIG. 2).

### Example 3—Application of Phenoage for Predicting Mortality Risk

It was subsequently demonstrated that when applying the resulting phenoage to a test set consisting of only cats that were not used during training of the algorithm, the difference between phenoage and chronological age (phenoage advance) was associated with a significant increase in mortality risk.

The magnitude of the effect was calculated to be a hazard ratio of 1.36 for a 1 year increase in phenoage compared to chronological age.

All publications mentioned in the above specification are herein incorporated by reference. Various modifications and variations of the disclosed methods, compositions and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A method for determining a mortality risk and/or probability of a healthy lifespan of a cat; said method comprising determining the level of one or more biomarker(s) in one or more samples obtained from the cat, wherein the one or more biomarker(s) is selected from the group consisting of white blood cell count, haemoglobin, serum urea nitrogen, serum AST, serum chloride, serum total bilirubin, serum globulin, haematocrit, red blood cells count, serum albumin, and mean corpuscular hemoglobin concentration.

2. A method for determining a mortality risk and/or probability of a healthy lifespan of a cat; said method comprising determining the level of one or more biomarker(s) in one or more samples obtained from the cat, wherein the one or more biomarker(s) is selected from the group consisting of white blood cell count, haemoglobin, serum urea nitrogen, serum AST, serum chloride, serum total bilirubin, serum globulin, and red blood cell count.

3. The method according to claim 1 wherein the biomarker is white blood cell count.

4. The method according to claim 1 wherein the biomarker is haemoglobin.

5. The method according to claim 1 wherein the biomarker is serum urea nitrogen.

6. The method according to claim 1 wherein the biomarker is serum AST.

7. The method according to claim 1 wherein the method comprises determining the level of each of white blood cell count, serum haemoglobin, serum urea nitrogen and serum AST.

8. The method according to claim 1 wherein the level of one or more further biomarkers selected from serum sodium, serum cholesterol, serum potassium, serum alkaline phosphatase, and/or serum GGT is determined in the one or more samples obtained from the cat.

9. The method according to claim 2 wherein the method comprises determining the level of each of white blood cell count, haemoglobin, serum urea nitrogen, serum AST, serum chloride, serum total bilirubin, serum globulin, red blood cell count, serum sodium, serum cholesterol, serum potassium, serum alkaline phosphatase, and serum GGT.

10. The method according to claim 1 wherein the one or more sample(s) is a blood sample.

11. The method according to claim 1 wherein the method further comprises combining the level of the one or more biomarker(s) with the chronological age of the cat.

12. A method for determining a mortality risk and/or probability of a healthy lifespan of a cat, said method comprising:
a. determining the level of the following biomarkers; white blood cell count, haemoglobin, serum urea nitrogen, serum AST, serum chloride, serum total bilirubin, serum globulin, red blood cell count, serum sodium, serum cholesterol, serum potassium, serum alkaline phosphatase, and serum GGT in one or more samples obtained from the cat; and
b. determining a phenotypic age (Phenoage) of the cat using formula (1):
$$Phenoage = ln ( \frac{{γ}_{2} * {e}^{xb} * \{ {e}^{γ * age} - 1 \}}{{e}^{{β}_{02}} * γ} + 1 ) * \frac{1}{{γ}_{2}}$$
where xb is the sum of the value of each biomarker(s), multiplied by their respective coefficients according to formula (2):
$$xb = {\sum }_{u = 1}^{p} {x}_{u} {β}_{u} + {β}_{0}$$
and
c. using the phenotypic age to determine a mortality risk and/or probability of a healthy lifespan for the cat.

13. The method according to claim 12 wherein determining that the phenoage of the cat is greater than its chronological age is indicative of a higher mortality risk and/or a reduced probability of a healthy lifespan.

14. The method according to claim 12 wherein determining that the phenoage of the cat is less than its chronological age is indicative of a lower mortality risk and/or an increased probability of a healthy lifespan.

15. The method according to claim 12 wherein the method is performed on one or more samples obtained before and after a time interval and determining if there has been a change in the mortality risk and/or probability of a healthy lifespan of the cat during the time interval.

16-38. (canceled)
